(19) United States
(12) Patent Application Publication
(10) Pub. No.: US 2004/0043488 A1
(43) Pub. Date: Mar. 4, 2004

(54) Adeno-associated viral gene-transfer vector system

(75) Inventors: Joseph C. Glorioso, Cheswick, PA (US); Xiao Xiao, Wexford, PA (US); David M. Krisky, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: University of Pittsburgh of the Commonwealth System of Higher Education, Pittsburgh, PA

(21) Appl. No.: 10/638,510

(22) Filed: Aug. 11, 2003

Related U.S. Application Data
(63) Continuation of application No. 09/506,301, filed on Feb. 17, 2000, now abandoned.
(60) Provisional application No. 60/120,391, filed on Feb. 17, 1999.

(51) Int. Cl. C12N 7/00; C12N 15/869

(52) U.S. Cl. 435/456; 435/235.1

(57) ABSTRACT

The present invention provides a recombinant HSV incorporating an adeno-associated virus (AAV) gene comprising a promoter and a polynucleotide sequence encoding a rep polypeptide. Preferably, the rep polypeptide or the promoter is conditionally active. The HSV can also include an AAV-derived ITR cassette and/or an AAV gene encoding a cap protein. The HSV can be used to direct site-specific integration of ITR cassettes within host cells and to facilitate packaging of such cassettes into AAV capsids.

### CROSS-REFERENCE TO RELATED PATENT APPLICATIONS

This patent application is a continuation of copending U.S. patent application Ser. No. 09/506,301, filed Feb. 17, 2000, which claims the benefit of U.S. Provisional Patent Application No. 60/120,391, filed Feb. 17, 1999.

### STATEMENT AS TO RIGHTS TO INVENTIONS MADE UNDER FEDERALLY SPONSORED RESEARCH AND DEVELOPMENT

This invention was made with support under grants 5R01DK49095-05, 5R01AR44526-02, and NO1-AR-6-2225 from the National Institutes of Health. The United States Government may have certain rights in this invention.

### TECHNICAL FIELD OF THE INVENTION

The present invention relates to a gene-transfer vector system based on Adeno-Associated Virus (AAV), more specifically a system employing Herpes Simplex Virus (HSV) for delivery of AAV-based vectors.

### BACKGROUND OF THE INVENTION

AAV is a replication-deficient parvovirus, which consists of a single strand of DNA encased in a proteinaceous capsid. AAV capsids can infect a wide variety of animal species and cell types, and over 40 serotypes are known to infect humans (see, e.g., M. Horwitz, “Adenoviridae and Their Replication”, 2d edition, B. N. Fields (ed.), Raven Press, Ltd., New York, Chapter 60, pp. 1679-1721 (1990)).

Infection begins when a capsid binds a host cell and delivers the AAV DNA into the cell, such that the DNA becomes integrated into the host cell chromosomes (typically chromosome 19 in human host cells). Thus integrated into the chromosomes, the virus persists in a latent state, such infection being silent, asymptomatic, and indefinite. Further progress through the AAV life cycle requires the products of “helper” genes, naturally supplied by superinfection of the AAV-infected cell with a helper virus (typically an adenovirus or a herpesvirus). While the products of the helper genes permit important roles in the life cycle of the helper virus, such products also cause the AAV DNA to be “rescued” from the host cell chromosome, and they permit replication of the rescued AAV DNA and packaging of the replicated DNA into AAV capsids. While the life cycles of adenoviruses and herpesviruses differ, superinfection by either type of helper virus ends with cell lysis and release of the helper virus and mature AAV capsids containing AAV DNA.

The AAV genome has been extensively dissected and sequenced (see, e.g., Srivastava et al., J. Virol., 45, 555-64 (1983)), and the function of each of its genes is now well understood. The AAV genome is a roughly 4.7 kb single-stranded DNA fragment and can be either positive or negative (either form is infectious). At each end of the genome is a 145 bp inverted terminal repeat (ITR), the first 125 bp of which can form Y- or T-shaped duplex structures to stabilize the genome during its life cycle. Additionally, the ITRs include cis-acting sequences directing viral DNA replication (ori), packaging (pkg) and host cell chromosome integration (int). Contained within the roughly 4.5 kb of AAV DNA positioned between the ITRs are two genes, which are expressed in the presence of helper gene products. Each of the two AAV genes encodes a series of polypeptides (rep and cap) through alternate splicing and/or differential promoters. The four rep polypeptides (rep78, rep68, rep62 and rep40) mediate replication and rescue of the AAV genome. For example, rep78/rep68 specifically bind to the ITRs, mediate site-specific end nuclease cleavage at the terminal resolution site, and also potentiate DNA-DNA and DNA-RNA helicase activities. In addition, the rep proteins regulate transcription from viral promoters and also mediate targeted integration into host chromosomes. The cap polypeptides (VP1, VP2 and VP3) form the virion capsid.

Many properties of AAV (e.g., its stability, its ability to stably integrate chromosomes without the need for replication, its broad host range, etc.) are attractive features for gene transfer applications. Indeed, as AAV DNA is infectious as plasmid DNA, construction of AAV-based vectors is quite feasible. Moreover, because each of the cis-acting functions directing replication, rescue, and packaging is localized to the ITRs, AAV-based vectors in which the intervening 4.5 kb DNA is missing (e.g., an “ITR cassette” including two AAV ITRs flanking a non-ITR sequence) are fully able to be replicated and packaged into AAV capsids and to infect host cells as wild-type viruses (provided the rep and cap polypeptides are supplied within a packaging cell). Moreover, as such AAV-derived vectors lack the rep and cap genes, they are not rescued from host cell chromosomes upon superinfection.

While many properties of AAV seemingly render it a superior vector platform for gene transfer applications, consistent production of high titer stocks of recombinant AAV currently is not feasible, especially on a large scale. Typically, such methods require supplying the rep and cap gene products in trans during packaging of the AAV-derived vector. One commonly employed method involves transfecting the desired ITR cassette into packaging cells followed by co-infection with wild-type AAV and an adenovirus. While it produces the desired AAV-derived vector, this method also produces unacceptably high levels of wild-type AAV. To avoid contamination with wild-type AAV, the rep and cap genes can be supplied on a second plasmid (separate from the AAV-derived vector) that is co-transfected into packaging cells with the vector plasmid. Because such methods depend on the kinetics of independent DNA transfer events to identical cells, they cannot consistently produce high titer AAV stocks, nor are they suitable for large scale production. While a packaging cell line stably expressing rep and cap genes can potentially eliminate the need for one transfection event, the use of such cell lines does not appreciably boost the titer of resulting AAV-derived vectors (see, e.g., Dutton, Genetic Engineering News, 14(1), 14-15 (1994) (reporting a titer of only about 10³ infectious units/ml)).

U.S. Pat. No. 5,856,152 describes a hybrid adenovirus-AAV vector including an ITR cassette within an adenovirus genome. While the system is apparently able to produce high-titer AAV stocks, it suffers from a number of drawbacks chiefly attributed to the properties of adenoviruses. For example, adenoviruses can be manipulated to carry only up to about 7.5 kb of exogenous DNA. Thus, where the rep and cap genes are introduced into the adenoviral genome, the carrying capacity of the ITR cassette is diminished. Deleting certain genes from the adenoviral genome can increase the carrying capacity of the vector; however, such gene products must be supplied in trans either to support adenoviral growth or to provide sufficient helper function to produce the desired AAV vector. Of course, such steps require either novel cell lines or secondary transfections to supply the deleted adenoviral genes, manipulations that ted to reduce AAV titer, as described above.

International Patent Application No. WO 98/21345 describes a hybrid AAV-amplicon vector including an ITR cassette and rep gene within a herpesviral amplicon genome. Such technology permits a vector size of only about 13 kb (packaged as concatamers) without requiring a helper HSV virus. Moreover, helper-free replication of amplicons requires cotransfection of a number of plasmids (or cosmids) to provide helper function, which reduces AAV titer, as described above. Where a helper HSV virus is employed to propagate the hybrid AAV-amplicon vectors, however, the resulting stock of hybrid vectors is contaminated with helper HSV viruses.

In view of the foregoing problems, there exists a need for an improved AAV-based gene-transfer vector system. Specifically, there is a need for a system for producing and/or delivering AAV-derived ITR cassettes that expands the amount of genetic material such vectors can effectively transfer while permitting efficient and consistent production of high-titer vector stocks.

### BRIEF SUMMARY OF THE INVENTION

The present invention provides a recombinant HSV incorporating an adeno-associated virus (AAV) gene comprising a promoter and a polynucleotide sequence encoding a rep polypeptide (“rep gene”). Preferably, the rep polypeptide or the promoter is conditionally active. The HSV can also include an AAV-derived ITR cassette and/or an AAV gene encoding a cap protein. The HSV can be used to direct site-specific integration of ITR cassettes within host cells and to facilitate packaging of such cassettes into AAV capsids.

The vector system is useful for the production of AAV vectors without requiring multiple transfection events within packaging cells, thus permitting the production of high titer AAV stocks. Moreover, the vector system permits relatively large transgene insertion within ITR cassettes as compared to currently available technology. These and other advantages of the present invention, as well as additional inventive features, will be apparent from the following detailed description.

### DETAILED DESCRIPTION OF THE INVENTION

The inventive AAV-based gene-transfer vector system employs an HSV for delivery or growth of AAV vectors. The HSV viral genome is well characterized, as is its life cycle, and the functions of more than 80 native HSV genes are largely defined. As roughly half of these genes are dispensable for growth in cell culture, large segments of the HSV genome can be deleted to accommodate transgenic material (Glorioso et al., in Viral Vectors, Academic Press, New York (Kaplitt & Loewy, eds.) 1-23 (1995)). Theoretically, up to about 30 kb of the HSV genome can thus be replaced with exogenous material without requiring complementary host cells for propagation.

In the context of the present invention, “HSV” refers to any Herpes Simplex Virus strain but excludes amplicons derived from herpesviruses. Thus, the HSV component of the invention includes at least an HSV-derived origin of replication (to permit the vector to replicate in permissive cells as a herpesvirus (see McGoech et al., Nuc. Acids Res., 14, 1727 (1986))), an HSV-derived packaging signal (to permit the vector to be packaged as an HSV (see Davidson et al., J. Gen. Virol., 55, 315 (1981))), and sufficient machinery to permit the virus to replicate within permissive cells without the need for a helper HSV or plasmid sequences.

While the HSV for use in the present invention is not an amplicon-based system, it can contain one or more mutations in HSV genes. Indeed, it is preferred that the vectors contain mutations in one or more genes essential for HSV replication so that such vectors are constrained to replicate as HSV viruses only in permissive cells. Any such mutation can be introduced into the HSV genome, many of which are known in the art (see, e.g., DeLuca, et al., J. Virol., 56, 558-70 (1985), Samaniego et al., J. Virol. 69(9), 5705-15 (1996); Field et al., J. Hygiene, 81, 267-77 (1978); Cameron et al., J. Gen. Virol., 69, 2607-12 (1988); Fink et al., Hum. Gene Ther., 3, 11-19, (1992); Jamieson et al., J. Gen. Virol., 76, 1417-31 (1995); Chou et al., Science, 250, 1262-66 (1990); Sears et al., J. Virol., 55, 338-46 (1985), U.S. Pat. Nos. 5,658,724 and 5,804,413, and International Patent Application WO 98/15637). Such mutations can, for example, affect one a combination of immediate early, early, or late genes, or a combination thereof. Desirably, the HSV backbone contains deficiencies in one or more essential genes to reduce toxicity within packaging and host cells (see, e.g., U.S. Pat. Nos. 5,879,934, 5,804,413, and 5,658,724, all to DeLuca).

While any mutation (or plurality of mutations) inactivating HSV replication in nonpermissive cells can be introduced into the genome, where the vector is to be employed for replicating AAV ITR cassettes (as described herein), the vector should retain sufficient helper function to permit replication of the AAV portion of the vector, desirably expressing early gene products. Thus, a preferred vector has an inactivating mutation in ICP27 because such vectors are unable to replicate efficiently in cells not complementing the HSV ICP27 protein; however, such vectors supply sufficient helper function for production and growth of AAV vectors in packaging cells, desirably immediate early and early gene functions). Such HSV backbones can be deficient for genes encoding ICP4 and ICP27 and packaged in a cell line complementing ICP4 and ICP27. Even more preferably, the HSV vector is deficient for UL41 and/or ICP22 as well, which further reduce cytotoxicity. Another preferred vector lacks functional ICP4, ICP22, and ICP27 genes, and can optionally lack a functional ICP0 gene.

Aside from having an HSV backbone as described immediately above, the inventive HSV vector incorporates a sequence derived from AAV, specifically at least one rep gene (i.e., including a promoter and a polynucleotide sequence encoding a rep polypeptide). The encoded rep protein can be any of the four AAV rep proteins, and the inventive virus can have polynucleotides encoding more than one rep protein (such as three or even all rep proteins). An encoded rep protein can be derived from any serotype of AAV, and where more than one rep genes are present, each can be derived from the same or different serotype of AAV. Moreover, an encoded rep protein can be a wild-type rep protein or a mutant form of such a protein, so long as the encoded rep protein possesses at least one activity of a corresponding wild-type rep protein, and most desirably the ability to direct site-specific integration of ITR cassettes into host cell chromosomes. Thus, the sequence can be modified, for example by removing splice signals or other regulatory elements, to encode only the desired rep protein. Additionally, the gene can encode an insertion, deletion, or substitution mutant form of a native AAV rep polypeptide, or an active fragment of a rep polypeptide. Preferably, any substitution is conservative in that it minimally disrupts the biochemical properties of the encoded rep polypeptide. Thus, where mutations are introduced to substitute amino acid residues, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues.

Within the inventive HSV, a polynucleotide encoding a rep protein is operably linked to a promoter suitable for driving its expression within host cells. For expression in many cell types, such a coding polynucleotide can be operably linked to its native promoters (i.e., the AAV p5 or p19 promoters). Alternatively, such a coding polynucleotide can be linked to other promoters many of which are known in the art. Examples of suitable promoters include prokaryotic promoters and viral promoters (e.g., retroviral ITRs, LTRs, immediate early viral promoters (IEp), such as herpesvirus IEp (e.g., ICP4-IEp and ICP0-IEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit β-globin regulatory elements), constitutively active promoters (e.g., the β-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context.

Such AAV-derived sequences can be introduced into any desired locus of the HSV backbone by any suitable method, many of which are well-known in the art. A common method of manipulating the HSV genome employs a host cell line to direct homologous recombination between a source HSV and a mutating vector (e.g., a plasmid, HSV or other viral vector, etc.) carrying the AAV-derived sequences flanked by sequences homologous to the desired locus within the HSV genome. A single round of homologous recombination within the host cell line can introduce one or several desired mutations into the source HSV, and the resultant viruses can be identified by Southern blotting, assaying for expression of a transgene, or other standard method. Other methods of manipulating the HSV genome are discussed in U.S. Pat. No. 5,998,174.

While the inventive HSV can serve as a helper virus for AAV replication, by virtue of the rep gene(s) it also can be employed to assist in excision/integration of AAV-derived ITR cassettes within host cells, and the invention provides a method for achieving such excision/integration within host cells. In the context of the present invention, an “ITR cassette” includes two sequences derived from an AAV ITR. Such sequences include an entire (or substantially an entire) ITR sequence, although some degree of minor and routine modification of a native ITR sequence is permissible, so long as it remains recognizable to one or more of the rep protein(s). Such sequences can be derived from any serotype of AAV, which enables the invention to be employed in a wide variety of host cells (i.e., any host animal able to be infected by some serotype of AAV). Thus, the AAV sequences of the inventive vectors preferably are selected to be compatible with the desired host. Within an ITR cassette, the two sequences derived from an AAV ITR flank the DNA which is to be transferred into the genome of the host cell. Typically, the DNA to be transferred is a transgene. Such a transgene is minimally a coding sequence for transcription operably linked to a promoter sequence for driving the transcription of the coding sequence. However, a transgene can include more than one coding sequence, and a transgene can optionally include other elements, such as polyadenylation sequences, ribosome entry sequences, transcriptional regulatory elements (e.g., enhancers, silencers, etc.), or other sequences. However, in some applications, the DNA to be transferred within the ITR cassette does not include a coding sequence. For example, the DNA can be a genetic marker, a consensus protein binding site, a ribozyme, antisense sequences, etc.

Through the use of the inventive HSV, the invention provides a method of directing site-specific integration of an AAV-derived ITR cassette into a desired target DNA molecule, such as a chromosome within a host cell. In accordance with this method, the ITR cassette and the inventive HSV are introduced into the host cell. Expression of the rep gene(s) within the cell so as to deliver the active encoded rep protein(s) within the cell an effect excision of the ITR from the vector and, desirably, integration of the ITR cassette within the desired target DNA molecule. Of course, where the ITR is introduced into the cell within a larger polynucleotide vector (e.g., an extrachromosomal polynucleotide such as a plasmid or virus), the method further effects excision of the cassette from the vector. By virtue of the aforementioned inactivation of essential HSV genes, the method can facilitate the safe delivery of AAV-derived ITR cassettes for use in populations of host cells, which can be in vivo or in vitro. For example, the method can be employed to deliver genes to isolated CD34+ lymphocytes in vitro which can then be employed in immunological protocols. An exemplary in vivo application could involve efficient delivery of active genes (e.g., encoding cytokines, a suicide gene, or other bioactive compound with antitumor activity) to dividing cells within a tumor Additionally, where such host cells are mitotically-active, the ITR cassette (having integrated into the chromosomal DNA) will be retained by successive generations of mitotic offspring, whereas the HSV backbone will not, by virtue of its inability to replicate in the absence of the essential HSV genes.

While the invention can be employed to direct integration of ITR cassettes from any source, it is even more useful when the ITR cassette is delivered to the cells in the same vector as the rep gene(s), as this ensures that the rep activity and the ITR cassettes are delivered to the same cells within a population. Thus, the inventive HSV vector can, and preferably does, contain an ITR cassette in addition to the rep gene(s). It will be appreciated that this embodiment can facilitate both delivery of the cassette to the cell (through HSV infection) and stable integration of the cassette into the cellular chromosomal DNA (via rep activity within the host cell). Additionally, due to the large capacity of the HSV genome to accommodate foreign DNA, this embodiment permits the delivery of relatively large AAV-derived ITR cassettes as compared to current technology. Thus, in some applications, the ITR cassette can include more than about 5 kb (e.g., more than about 10 kb), or even more than about 15 kb. Indeed, the inventive vector is able to deliver an ITR cassette including more than about 20 kb, the maximal size of the ITR being dictated by the available capacity of the HSV backbone to accommodate DNA (e.g., more than about 30 kb). The ability of the inventive HSV to deliver such large stretches of DNA as ITR cassettes thus permits even large mammalian genes to be delivered to cells, and it also facilitates tandem delivery of multiple genes within ITR cassettes.

Where the inventive HSV includes an ITR cassette, preferably any AAV-derived genes (and more preferably all such genes) are not within the ITR cassette to mitigate the potential for autologous rescue of the ITR cassette from the host cell chromosome. Also, within such a recombinant HSV, preferably, at least one rep gene is conditionally active (i.e., relatively more active under an identifiable permissive condition and relatively less under an identifiable nonpermissive condition). Desirably, the gene is only negligibly active (e.g., reduced by at least about 2 orders of magnitude, or even at least as much as about 3 orders of magnitude, such as at least about 4 orders of magnitude) under the nonpermissive condition, and ideally it is inactive under such conditions. Preferably, the nonpermissive conditions exist during HSV packaging, while the permissive conditions exist within the desired host cells. This arrangement minimizes (or even prevents) unwanted premature excision of the ITR cassettes from the HSV genomes during packaging, thus enhancing the number of produced HSV vectors that contain the cassettes. Of course, because the rep gene(s) is/are active in the desired target cells, excision and integration can proceed as described above.

Any suitable strategy for effecting conditional gene activity can be employed. For example, in one embodiment, the rep coding polynucleotide within the gene is operably linked to a regulable promoter (i.e., a promoter which is relatively more active under a permissive condition and less active under a nonpermissive condition). Many suitable regulable promoters are known in the art, enabling an artisan to select a particular promoter suitable for a desired use. Examples of such promoters include tissue-specific promoters (those active in epidermal tissue, dermal tissue, tissue of the digestive organs (e.g., cells of the esophagus, stomach, intestines, colon, etc., or their related glands), smooth muscles, such as vascular smooth muscles, cardiac muscles, skeletal muscles, lung tissue, hepatocytes, lymphocytes, endothelial cells, sclerocytes, kidney cells, glandular cells (e.g., those in the thymus, ovaries, testicles, pancreas, adrenals, pituitary, etc.), tumor cells, cells in connective tissue, cells in the central nervous system (e.g., neurons, neuralgia, etc.), cells in the peripheral nervous system, and other cells of interest), inducible promoters (e.g., active in the presence of factors such as antibiotics or immunosuppressive agents (e.g., tetracycline, rapamycin, etc.), hormones, heavy metals (e.g., the metallothionein promoter), cytokines (e.g., interferon-γ, interleukin-1, etc.), natural or artificial steroids (e.g., RU486), etc.), repressible promoters (inactive in the presence of similar agents). In other embodiments, conditional activity of the gene hinges on the conditional activity of the encoded rep protein. For example, the polynucleotide within the gene can encode a temperature-sensitive rep mutant polypeptide, or a mutant form of rep that retains high activity only in the presence of certain chemical species (e.g., magnesium). Several such proteins are known in the art (see, e.g., Gavin et al., J. Virol., 73(11), 9433-45 (1999)), and it is within the ordinary skill to isolate other conditionally active rep proteins. While conditional activity of the rep gene within the inventive HSV can be effected through the promoter or the coding polynucleotide, for even tighter control over the activity of the gene, preferably both approaches are employed in the same rep gene.

In another embodiment, in addition to the rep gene(s), the recombinant HSV vector also includes a second gene (or multiple genes) encoding all three AAV cap proteins to permit the packaging of an ITR cassette within an AAV capsid. Such encoded cap proteins can be wild-type or mutant isoforms, as described above with reference to the rep proteins. Where the HSV has such cap genes, the invention provides a method of packaging ITR cassettes within AAV viruses. The ITR cassette and the inventive HSV are delivered to a host cell as described above. Within the cell, the cap gene(s) are expressed to produce operable cap polypeptides. The rep proteins facilitate excision of the ITR cassette from any vector into which it has integrated, and the cap polypeptides effect packaging of the ITR cassette as an AAV. While the cap gene(s) can be introduced into the cell on any vector, preferably the gene(s) is(are) introduced on the same vector used to deliver the rep gene(s). Such a method will also produce HSV vectors. However, the resulting AAV capsids can be purified from HSV by detergent treatment, which destroys HSV but does not affect AAV. Alternatively, the two types of viruses can be separate using a cesium chloride gradient, from which the AAV viruses can be isolated and purified. Additionally, in performing the inventive method, multiple copies of the rep and cap genes can be introduced into the packaging cell to amplify the amount of such polypeptides produced within the cell. A greater amount of such polypeptides can boost the titer of AAV produced.

Generally, the inventive recombinant HSV is most useful when enough of the virus can be delivered to a cell population to ensure that the cells are confronted with a predefined number of viruses. Thus, the present invention provides a stock, preferably a homogeneous stock, of HSV. The preparation and analysis of HSV stocks is well known in the art. Viral stocks vary considerably in titer, depending largely on viral genotype and the protocol and cell lines used to prepare them. Preferably, such a stock has a viral titer of at least about 10⁵ plaque-forming units (pfu), such as at least about 10⁶ pfu or even more preferably at least about 10⁷ pfu. In still more preferred embodiments, the titer can be at least about 10⁸ pfu, or at least about 10⁹ pfu, and high titer stocks of at least about 10¹⁰ pfu or at least about 10¹¹ pfu are most preferred.

For delivery into a host (such as a human), a recombinant HSV according to the present invention (or stock of such viruses) can be incorporated into a suitable carrier. As such, the present invention provides a composition comprising a recombinant HSV of the present invention and a suitable carrier, particularly a pharmacologically or physiologically acceptable carrier. Such compositions can be formulated in a conventional manner using one or more pharmacologically or physiologically acceptable carriers comprising excipients, as well as optional auxiliaries which facilitate processing of the active compounds into preparations which can be used pharmaceutically. Proper formulation is dependent upon the route of administration chosen. Thus, for systemic injection, the recombinant HSV can be formulated in aqueous solutions, preferably in physiologically compatible buffers. For transmucosal administration, penetrants appropriate to the barrier to be permeated are used in the formulation. Such penetrants are generally known in the art. For oral administration, the recombinant HSV can be combined with carriers suitable for inclusion into tablets, pills, dragees, capsules, liquids, gels, syrups, slurries, diposomes, suspensions and the like. For administration by inhalation, the recombinant HSV conveniently is delivered in the form of an aerosol spray presentation from pressurized packs or a nebulizer, with the use of a suitable propellant. The recombinant HSV can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. For application to the skin, the recombinant HSV can be formulated into a suitable gel, magma, creme, ointment, or other carrier. For application to the eyes, the recombinant HSV can be formulated in aqueous solutions, preferably in physiologically compatible solution (e.g., a pH-buffered saline solution). The recombinant HSV can also be formulated into other pharmaceutical compositions such as those known in the art.

### EXAMPLES

While one of skill in the art is fully able to practice the instant invention upon reading the foregoing detailed description, the following examples will help elucidate some of its features. In particular, they demonstrate the ability of a recombinant HSV to supply essential AAV genes to cells, and the ability of an HSV containing an AAV-derived ITR cassette and an AAV rep gene to deliver the cassette to targeted cells. Of course, as these examples are presented for purely illustrative purposes, they should not be used to construe the scope of the invention in a limited manner, but rather should be seen as expanding upon the foregoing description of the invention as a whole.

The procedures employed in these examples, such as HSV vector construction, molecular cloning techniques, Southern hybridization, cell culture, and virus growth and production, are familiar to those of ordinary skill in this art (see, e.g., Sambrook et al., “Molecular Cloning: A Laboratory Manual,” 2d edition, Cold Spring Harbor Press (1989); U.S. Pat. No. 5,998,174). As such, and in the interest of brevity, experimental details are not recited in detail.

### Example 1

This example demonstrates the ability of a recombinant HSV to supply essential AAV genes to cells.

Using standard techniques, a plasmid was constructed placing an expression cassette containing the AAV rep and cap coding sequences operably linked in tandem to the AAV p5 promoter. Using a novel Pac1 restriction site, this construct was introduced into the UL41 locus of an HSV mutant deleted for ICP4 and ICP27. The resulting HSV lacked functional HSV ICP4, ICP27 and UL41 genes and had both the AAV rep and cap coding polynucleotides under transcriptional control of the AAV p5 promoter. This genotype was confirmed by Southern hybridization.

To test whether the recombinant HSV could supply rep and cap function, a stock of the HSV was used to infect HEK 293 cells, which had been transfected with a plasmid containing an AAV genome. Subsequently, the cells were superinfected with adenovirus to supply helper function, and the production of mature AAV viruses was assessed by standard methods. While the yield of AAV was small (about 0.1 pfu/cell), this result demonstrated that the rep and cap genes present within the HSV were functional.

### Example 2

This example demonstrates the construction of a recombinant HSV containing a conditionally active AAV-derived rep gene.

Using standard techniques, a plasmid was constructed placing an expression cassette containing a coding polynucleotide encoding a temperature sensitive AAV rep (^{ts}rep) protein operably linked to the AAV p5 promoter. The encoded protein is relatively less active at human core body temperatures, but is more active during culture conditions (see, e.g., Gavin et al., supra). This construct is inactive at 32-33° C. and active at 37-39° C. Using a novel Pac1 restriction site, this construct was introduced into the HSV1 genome deleted for ICP4 and ICP27 at the UL41 locus as described in Example 1. The resulting HSV lacked functional HSV ICP4, ICP27 and UL41 genes and had the ^{ts}rep coding polynucleotides under transcriptional control of the AAV p5 promoter. This genotype was confirmed by Southern hybridization.

### Example 3

This example demonstrates the construction of a recombinant HSV containing a conditionally active AAV-derived rep gene.

Similarly to the construct described in Example 2, a plasmid can be constructed to place an expression cassette containing polynucleotide encoding a AAV rep protein operably linked to the a minimal Gal4-TATA promoter (see, e.g., Ji et al., Gene Ther., 6(3), 393-402 (1999), and references cited therein). The expression cassette is active in the presence of chimeric transcription factors that bind Gal4 sequences (e.g., chimeric Gal4/VP16 proteins, see Wang et al., Gene Ther., 4(5):432-41 (1997); Wang et al., Proc. Nat. Acad. Sci. (USA), 91(17), 8180-84 (1994)). This construct can be introduced into the HSV1 genome deleted for ICP4 and ICP27 at the UL41 locus as described in Example 2. The resulting HSV will lack functional HSV ICP4, ICP27 and UL41 genes and have the re coding polynucleotide under transcriptional control of the inducible promoter.

### Example 4

This example demonstrates the ability of an HSV containing an AAV-derived ITR cassette and an AAV rep gene to deliver the cassette to targeted cells.

A construct containing the E. coli lacZ coding sequence operably linked to the HSV ISP0 promoter was inserted by Pac1 mutagenesis into the UL41 locus of an HSV strain deficient for ICP4 and ICP27. A second construct was created in which green fluorescence protein (GFP) and neomycin resistance (Neo^{r}) genes were inserted between two AAV-derived ITR sequences to form an ITR cassette. To introduce this second construct into the HSV genome, it will be cloned into a plasmid between sequences homologous to the HSV UL23 locus, which will then be employed within a cell to introduce the construct into the HSV genome by homologous recombination. The resultant HSV will lack functional HSV ICP4, ICP27, UL41 and thymidine kinase genes and it will possess the LacZ gene and ITR cassette, which can be confirmed by Southern hybridization.

The HSV will then be crossed by homologous recombination with any of the viruses described in Examples 2-3 to produce a virus having both the ITR cassette and the conditionally active rep gene. Successful recombinants will produce white plaques (indicating loss of the lacZ gene); that also have GFP activity (indicating presence of the ITR cassette), presence of both AAV sequences can be further confirmed by Southern hybridization. Of course, the mature HSV vectors are packaged under nonpermissive conditions for the conditionally active rep gene (e.g., at nonpermissive temperature for the ^{ts}rep mutant or in the absence of the Gal4/VP16 transcriptional activator where the minimal Gal4-TATA promoter is employed).

After purification, the resultant recombinant HSV viruses are introduced into desired target cells under permissive conditions for the conditionally active rep gene (e.g., in the presence of the transcriptional activator or at permissive temperatures). Under such conditions, the rep protein will function to excise the ITR cassette and, desirably, to direct integration into the host cell chromosome.

### Incorporation by Reference

All sources (e.g., inventor's certificates, patent applications, patents, printed publications, repository accessions or records, utility models, World-Wide Web pages, and the like) referred to or cited anywhere in this document or in any drawing, Sequence Listing, or Statement filed concurrently herewith are hereby incorporated into and made part of this specification by such reference thereto.

### Guide to Interpretation

The foregoing is an integrated description of the invention as a whole, not merely of any particular element of facet thereof. The description describes “preferred embodiments” of this invention, including the best mode known to the inventors for carrying it out. Of course, upon reading the foregoing description, variations of those preferred embodiments will become obvious to those of ordinary skill in the art. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

As used in the foregoing description and in the following claims, singular indicators (e.g., “a” or “one”) include the plural, unless otherwise indicated. Recitation of a range of discontinuous values is intended to serve as a shorthand method of referring individually to each separate value falling within the range, and each separate value is incorporated into the specification as if it were individually listed. As regards the claims in particular, the term “consisting essentially of” indicates that unlisted ingredients or steps that do not materially affect the basic and novel properties of the invention can be employed in addition to the specifically recited ingredients or steps. In contrast, the terms “comprising,” “having,” or “incorporating” indicate that any ingredients or steps can be present in addition to those recited. The term “consisting of” indicates that only the recited ingredients or steps are present, but does not foreclose the possibility that equivalents of the ingredients or steps can substitute for those specifically recited.

What is claimed is:

1. A recombinant herpes simplex virus (HSV) comprising a rep gene, which comprises a promoter operatively linked to a polynucleotide encoding an adeno-associated virus (AAV) rep polypeptide, wherein the rep polypeptide is conditionally active.

2. The recombinant HSV of claim 1, wherein the rep polypeptide is obtained from an AAV rep78, rep68, rep62, or rep40 protein.

3. The recombinant HSV of claim 1, wherein the rep polypeptide is an AAV rep78 protein.

4. The recombinant HSV of claim 1, wherein the rep polypeptide is an AAV rep68 protein.

5. The recombinant HSV of claim 1, wherein the rep polypeptide is an AAV rep62 protein.

6. The recombinant HSV of claim 1, wherein the rep polypeptide is an AAV rep40 protein.

7. The recombinant HSV of claim 1, further comprising an Intermediate Terminal Repeat (ITR) cassette, which comprises two AAV-derived ITR sequences flanking a non-ITR polynucleotide.

8. The recombinant HSV of claim 7, wherein the rep gene is not within the ITR cassette.

9. The recombinant HSV of claim 1, further comprising a cap gene comprising a promoter operatively linked to a polynucleotide sequence encoding an AAV cap polypeptide.

10. The recombinant HSV of claim 9, further comprising an ITR cassette, which comprises two AAV-derived ITR sequences flanking a non-ITR polynucleotide.

11. The recombinant HSV of claim 10, wherein the rep gene is not within the AAV ITR cassette.

12. The recombinant HSV of claim 1, which is deficient for at least one essential HSV.

13. The recombinant HSV of claim 12, wherein the essential HSV gene is an immediate early, early or late HSV gene.

14. The recombinant HSV of claim 12, wherein the essential HSV gene is ICP27.

15. The recombinant HSV of claim 1, wherein the promoter is conditionally active.

16. The recombinant HSV of claim 1, wherein the promoter is a tissue specific promoter.

17. The recombinant HSV of claim 1, wherein the promoter is an HSV promoter.

18. The recombinant HSV of claim 1, which is replication incompetent in cells other than packaging cells.

19. A viral stock comprising the recombinant HSV of claim 1.

20. A composition comprising the recombinant HSV of claim 1 and a physiologically-acceptable carrier.

21. The composition of claim 20, which further comprises an ITR cassette.

22. The composition of claim 21, wherein the ITR cassette is within an HSV vector.

23. The composition of claim 20, further comprising a second HSV that comprises an ITR cassette.

24. A recombinant herpes simplex virus (HSV) comprising a rep gene, which comprises a promoter operatively linked to a polynucleotide encoding an adeno-associated virus (AAV) rep polypeptide, a cap gene, which comprises a promoter operatively linked to a polynucleotide sequence encoding an AAV cap polypeptide, and an Intermediate Terminal Cassette (ITR) cassette, which comprises two AAV-derived ITR sequences flanking a non-ITR polynucleotide, wherein the rep polypeptide or the promoter is conditionally active.

25. The recombinant HSV of claim 24, wherein the rep polypeptide is obtained from an AAV rep78, rep68, rep62, or rep40 protein.

26. The recombinant HSV of claim 24, wherein the rep polypeptide is an AAV rep78 protein.

27. The recombinant HSV of claim 24, wherein the rep polypeptide is an AAV rep68 protein.

28. The recombinant HSV of claim 24, wherein the rep polypeptide is an AAV rep62 protein.

29. The recombinant HSV of claim 24, wherein the rep polypeptide is an AAV rep40 protein.

30. The recombinant HSV of claim 24, wherein the rep gene is not within the AAV ITR cassette.

31. The recombinant HSV of claim 24, which is deficient for at least one essential HSV gene.

32. The recombinant HSV of claim 31, wherein the essential HSV gene is an immediate early, early or late HSV gene.

33. The recombinant HSV of claim 31, wherein the essential HSV gene is ICP27.

34. The recombinant HSV of claim 24, wherein the promoter is conditionally active.

35. The recombinant HSV of claim 24, wherein the promoter is a tissue specific promoter.

36. The recombinant HSV of claim 24, wherein the promoter is an HSV promoter.

37. The recombinant HSV of claim 24, which is replication incompetent in cells other than packaging cells.

38. A viral stock comprising the recombinant HSV of claim 24.

39. A composition comprising the recombinant HSV of claim 24 and a physiologically-acceptable carrier.

40. The composition of claim 39, which further comprises an ITR cassette.

41. The composition of claim 40, wherein the ITR cassette is within an HSV vector.

42. The composition of claim 39, further comprising a second HSV that comprises an ITR cassette.

43. A recombinant herpes simplex virus (HSV) comprising a rep gene, which comprises a promoter operatively linked to a polynucleotide encoding an adeno-associated virus (AAV) rep polypeptide, wherein the rep polypeptide or the promoter is conditionally active, and wherein the promoter is an inducible promoter.

44. A recombinant herpex simplex virus (HSV) comprising a rep gene, which comprises a promoter operatively linked to a polynucleotide encoding an adeno-associated virus (AAV) rep polypeptide, wherein the rep polypeptide is active at a first permissive temperature, and inactive at a second nonpermissive temperature.
